(19) United States
(12) Patent Application Publication
(10) Pub. No.: US 2024/0042023 A1
(43) Pub. Date: Feb. 8, 2024

(54) Fc-Receptor CAR Constructs and Cells

(71) Applicant: ImmunityBio, Inc., Culver City, CA (US)

(72) Inventors: Laurent H. Boissel, Culver City, CA (US); Barry J. Simon, San Diego, CA (US); Wenjie Hu, Culver City, CA (US); Hans G. Klingemann, Culver City, CA (US)

(73) Assignee: ImmunityBio, Inc., Culver City, CA (US)

(21) Appl. No.: 18/258,925

(22) PCT Filed: Dec. 20, 2021
(86) PCT No.: PCT/US21/64408

§ 371 Date: Jun. 22, 2023

Related U.S. Application Data
(60) Provisional application No. 63/129,340, filed on Dec. 22, 2020.

(51) Int. Cl. A61K 39/00; C07K 14/725; C07K 16/28; A61P 35/00

(52) U.S. Cl. CPC A61K 39/4613; C07K 14/7051; C07K 16/283; A61P 35/00; A61K 39/4631; A61K 39/464411; A61K 2239/17; C07K 2319/03; A61K 2239/13; C07K 2317/622; A61K 2239/22

(57) ABSTRACT

Chimeric antigen receptors with an antibody-binding domain are presented that are preferably expressed from a recombinant cell in a therapeutic cell, and particularly in an NK-92 cell or derivative thereof. Notably, such modified cells have multiple modes of cytotoxicity, improved on-target cell killing, decreased off-target cell killing, show significant expression of the recombinant CAR, and/or increased CAR-mediated cytotoxicity.

This application claims priority to our copending U.S. Provisional Patent Application with the Ser. No. 63/129,340, which was filed Dec. 22, 2020, and which is incorporated by reference herein in its entirety.

### SEQUENCE LISTING

The content of the ASCII text file of the sequence listing named 104077.0021_REV005_ST25.txt, which is 144 KB in size, created on Dec. 16, 2021 and electronically submitted via EFS-Web along with the present application, is incorporated by reference herein in its entirety.

### FIELD OF THE INVENTION

The field of the invention is chimeric antigen receptors (CAR) and recombinant cells expressing such CARs, particularly as they relate to CARs with an antigen binding domain that binds an Fc portion of an antibody such as a CD16 Fc binding domain, a CD32 Fc binding domain, or a CD64 Fc binding domain.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In many instances, therapeutic antibodies can be used to effect antibody-dependent cell-mediated cytotoxicity (ADCC), which has become a promising avenue for immune therapy of various cancers using genetically modified NK cells. ADCC is mediated by the CD16 receptor that binds the therapeutic antibody and triggers granzyme and granulysin release towards the antibody-bound target cell. Unfortunately, however, expression of the CD16 receptor is often rapidly downregulated and so limits therapeutic utility of these antibodies using native NK cells. In addition, CD16 on native NK cells has a relatively low affinity to the Fc portion of an antibody, thus even further limiting therapeutic use. More recently, recombinant NK cells were developed (commercially available from NantKwest as haNK cells) that express a high affinity version of CD16, thus significantly increasing the therapeutic potential as the recombinant CD16 variant is not subject to downregulation and has a higher affinity towards the Fc portion of an antibody.

In another approach, a chimeric protein was produced that had a CD16 intracellular and transmembrane portion and a CD64 extracellular portion to so maintain ADCC capability with an increased affinity due to the CD64 portion (see e.g., Frontiers in Immunology, December 2018, Vol. 9, Article 2873) While conceptually attractive, various disadvantages remained. Among other things, in vivo antitumor activity has not been proven and the authors postulated that NK cells expressing CD64/16A could be less efficient at serial killing. In further attempts, as described in WO 2015/179833, hybrid constructs in which multimers of CD64 were coupled to a transmembrane domain and intracellular T-cell signaling domains to so arm T cells with a chimeric receptor were produced that could activate cytotoxic cell killing of T cells induced by antibody binding.

In still another approach, a chimeric antigen receptor was produced that included a CD16V domain coupled to various signaling domains as described in US 2018/0133252. Similarly, U.S. Pat. No. 7,618,817 described certain CAR constructs where a CD16 portion was used to provide the binding specificity in a CAR that was expressed from a retroviral construct in NK-92 cells. While such approaches led to recombinant cytotoxic cells that were able to bind antibodies, generation of such cell lines is often associated with loss or reduction of natural cytotoxicity, decreased on-target cell killing and/or increased off-target cell killing as compared to native NK cells, attenuated expression of the recombinant CAR, and/or decreased CAR-mediated cytotoxicity for at least some of the CAR constructs, even where such constructs are 2^{nd} or 3^{rd} generation CAR constructs.

Thus, even though various systems and methods of CARs that bind an Fc portion of an antibody are known in the art, all or almost all of them suffer from several drawbacks. Therefore, there remains a need for compositions and methods for improved CARs and CAR expressing cells that have multiple modes of cytotoxicity, improved on-target cell killing, decreased off-target cell killing, significant expression of the recombinant CAR, and/or increased CAR-mediated cytotoxicity.

### SUMMARY OF THE INVENTION

The inventive subject matter is directed to various compositions and methods of recombinant CARs and cells expressing such CARs where such cells exhibit multiple modes of cytotoxicity, improved on-target cell killing, decreased off-target cell killing, significant expression of the recombinant CAR, and/or increased CAR-mediated cytotoxicity.

In one aspect of the inventive subject matter, the inventors contemplate a recombinant chimeric antigen receptor (CAR) that comprises an antibody binding domain having an antibody-binding portion of a polypeptide having a sequence selected from the group consisting of SEQ ID NO:12, SEQ ID NO:16, SEQ ID NO:19, SEQ ID NO:22, SEQ ID NO:25, SEQ ID NO:28, and SEQ ID NO:31. In such CAR the antibody binding domain is further coupled to a polypeptide comprising in sequence an optional hinge portion, a transmembrane portion, and a signaling domain.

In selected embodiments, the antibody binding domain has a peptide sequence selected from the group consisting of SEQ ID NO:17, SEQ ID NO:20, SEQ ID NO:23, SEQ ID NO:26, SEQ ID NO:29, and SEQ ID NO:32. Typically, the hinge portion has a peptide sequence of SEQ ID NO:3, the transmembrane portion has a peptide sequence of SEQ ID NO:5, SEQ ID NO:74, SEQ ID NO:76, SEQ ID NO:78, SEQ ID NO:80, SEQ ID NO:82, or SEQ ID NO:84, and/or the signaling domain has a peptide sequence of SEQ ID NO:1. In further contemplated embodiments, the CAR may include at least one second signaling domain, which may be distinct from the initial the signaling domain. For example, in some embodiments the signaling domain has a peptide sequence selected from the group consisting of SEQ ID NO:7, SEQ ID NO:8, and SEQ ID NO:9.

In further exemplary embodiments, the antibody binding domain in such CARs has a peptide sequence selected from the group consisting of SEQ ID NO:17, SEQ ID NO:20, SEQ ID NO:23, SEQ ID NO:26, SEQ ID NO:29, and SEQ ID NO:32, and the signaling domain has a peptide sequence of SEQ ID NO:1. Preferably, the hinge portion has a peptide sequence of SEQ ID NO:3, and/or the transmembrane portion has a peptide sequence of SEQ ID NO:5, SEQ ID NO:74, SEQ ID NO:76, SEQ ID NO:78, SEQ ID NO:80, SEQ ID NO:82, or SEQ ID NO:84. Where desired, the CAR may further include at least one additional signaling domain (e.g., having the sequence of SEQ ID NO:1 or other signaling domain).

In further contemplated aspects, the inventors contemplate a recombinant nucleic acid that encodes the chimeric antigen receptors as presented herein. Preferably, but not necessarily, the nucleic acid is codon-optimized to human codon usage. Moreover, it is contemplated that the nucleic acid may also include a sequence portion that encodes a cytokine, a CD16, a homing receptor, and/or a TGF-beta trap. (all of which may be arranged in a polycistronic configuration). For example, the recombinant nucleic acid may be part of a lentiviral vector, or part of a DNA vector.

In still further contemplated aspects, the inventors contemplate a (typically mammalian) cell transfected with a recombinant nucleic acid as presented herein. For example, the cell is a NK cell (e.g., NK-92 cell, a genetically modified NK-92 cell, or an autologous NK cell) or a T cell. Viewed from a different perspective, the inventors also contemplate a recombinant NK cell (e.g., NK-92 cell, a genetically modified NK-92 cell, or an autologous NK cell) that is transfected with a recombinant nucleic acid encoding a recombinant chimeric antigen receptor as described herein.

Consequently, the inventors also contemplate a method of treating cancer in a patient in need thereof in which a therapeutically effective amount of the cells presented herein is administered to the patient, thereby treating the cancer. Most typically, about 1×10⁸ to about 1×10¹¹ cells per m² of body surface area of the patient are administered to the patient. In addition, at least one additional therapeutic entity may be administered to the patient such as a viral cancer vaccine, a bacterial cancer vaccine, a yeast cancer vaccine, N-803, an antibody, a stem cell transplant, and/or a tumor targeted cytokine. For example, cancers contemplated to be treated include leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

Thus, uses of cells as presented herein are contemplated in the treatment of cancer.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic illustration of various chimeric antigen receptors presented herein.

FIG. 2 depicts exemplary CD16 FACS scan results for aNK cells, haNK cells, and CD16-CAR 28.E cells.

FIG. 3 depicts exemplary results for cytotoxicity of CD16-CAR 28.E cells against NK sensitive K562 cells as compared to haNK cells.

FIG. 4 depicts exemplary results for ADCC activity of CD16-CAR 28.E cells against SUP-B15^{CD20+} cells in the presence and absence of on-target and off-target antibodies.

### DETAILED DESCRIPTION

The inventors have discovered various compositions and methods of recombinant CARs and cells expressing such CARs where such cells exhibit multiple modes of cytotoxicity, improved on-target cell killing, decreased off-target cell killing, significant expression of the recombinant CAR, and/or increased CAR-mediated cytotoxicity.

Based on the inventors' earlier discovery that various CAR constructs using FcεRIγ as the signaling domain in a first generation-type construct had improved expression and afforded increased target specific killing in NK-92 cells expressing such CAR constructs, the inventors set out to modify various cells, and particularly NK-92 cells and genetically modified NK-92 cells to arm such cells with high affinity CAR constructs that would bind the Fc portion of an antibody (and especially an IgG antibody) and so enhanced cytotoxic effects of cells expressing such CAR. Indeed, where these CARs were expressed on NK cells, ADCC of these cells was substantially increased over unmodified NK cells.

Unexpectedly, and as is described in more detail below, the so generated recombinant NK cells had multiple modes of cytotoxicity, improved on-target cell killing, decreased off-target cell killing, significant expression of the recombinant CAR, and/or increased CAR-mediated cytotoxicity when compared to unmodified NK cells, NK-92 cells, and in some cases even when compared to NK cells expressing CD16 and co-expressing an FcεRIγ signaling polypeptide or a CD3ζ chain (e.g., as taught in U.S. Pat. No. 9,181,322).

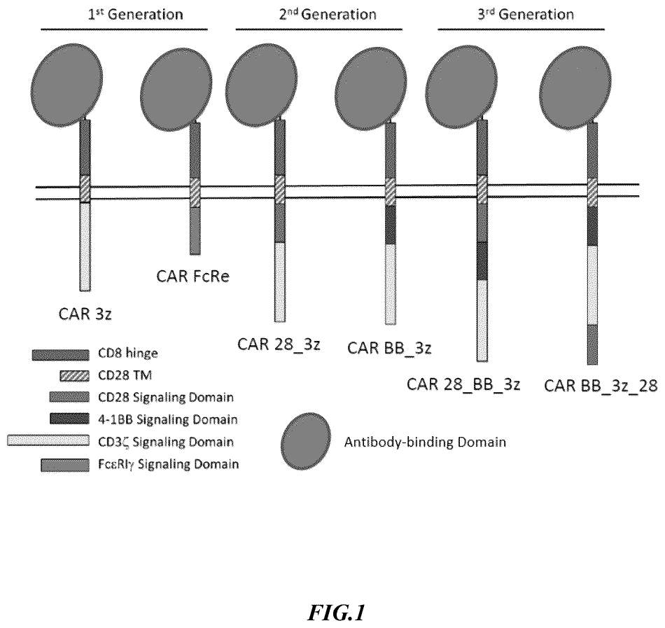

In preferred embodiments, the inventors contemplate CAR constructs that comprise in a single polypeptide chain an antibody binding domain that has an antibody-binding portion, followed in sequence by an extracellular optional hinge portion, a transmembrane portion, and an intracellular signaling domain. Depending on the particular use and/or arrangement of intracellular signaling domains, it should be appreciated that the so prepared CAR constructs can be 1^{st}, 2^{nd} or 3^{rd} generation CARs. FIG. 1 exemplarily depicts contemplated CARs useful in conjunction with the teachings presented herein. For example, 1^{st} generation CA constructs may comprise a single signaling domain such as a CD3ζ intracellular signaling domain, and more preferably a FcεRIγ signaling domain. Notably, where CAR constructs had 1^{st} generation architecture, such CARs with an FcεRIγ signaling domain had superior properties in NK cells as compared to other CAR constructs. Such finding was especially unexpected as heretofore known 1^{st} generation CARs in T cells had performed relatively poorly as compared to CARs that had a CD3ζ, a 4-1BB, or a CD28 signaling domain and optionally additional signaling domains as commonly found in second and third generation CARs. As will be readily appreciated, contemplated CARs may also include multiple FcεRIγ and/or CD3ζ intracellular signaling domains.

In further examples, the CAR construct may include at least two distinct intracellular signaling domains, and typical examples for such CAR constructs include those in which a CD3ζ intracellular signaling domain is coupled to a CD28 signaling domain or a 4-1BB signaling domain as is exemplarily depicted in the 2^{nd} generation CAR constructs of FIG. 1. Moreover, it is noted that contemplated CAR constructs may include more than two signaling domains (that are typically distinct), and exemplary 3^{rd} generation CAR constructs include those in which a CD3ζ intracellular signaling domain is coupled to a CD28 signaling domain and a 4-1BB signaling domain. Of course, it should be recognized that the particular sequence order of the intracellular signaling domains may vary, and all arrangements are deemed suitable for use herein.

With respect to the antibody binding domain, it is generally contemplated that the CARs presented herein have at least an antibody-binding portion that will bind to the Fc portion of an antibody, and most preferably the Fc portion of an IgG. However, in alternative aspects, the Fc portion may also belong to an antibody class other than IgG, including IgA, IgM, and IgE. Therefore, suitable antibody binding domains will preferably include the full-length polypeptides or antibody-binding portions of CD16A, CD16B, CD32A, CD32B, CD64A, CD64B, and CD64C, and in less preferred aspects also protein A and protein G. Therefore, suitable full-length polypeptides or antibody-binding portions will have or comprise an amino acid sequence according to SEQ ID NO:12, SEQ ID NO:16, SEQ ID NO:19, SEQ ID NO:22, SEQ ID NO:25, SEQ ID NO:28, and SEQ ID NO:31 (or portion of each of these sequences). Where the antibody-binding portion is an extracellular domain of CD16, CD32, or CD64, especially contemplated extracellular domains will have or comprise an amino acid sequence according to SEQ ID NO:17, SEQ ID NO:20, SEQ ID NO:23, SEQ ID NO:26, SEQ ID NO:29, and SEQ ID NO:32 (or portion of each of these sequences).

Regardless of the type of antibody binding domain, it should be appreciated that in most cases the antibody binding domain will be coupled to the transmembrane portion via a hinge portion to provide for flexibility of the antibody binding domain relative to the transmembrane portion. However, it should be recognized that in certain embodiments the hinge portion is omitted as is shown in some of the example sequences below. Where present, the hinge portion is most typically, but not necessarily, a short and flexible polypeptide having between about 5 and 100 (predominantly hydrophilic) amino acid residues. Therefore, suitable high portions specially include a CD8 hinge portion (especially human CD8 hinge portion) having or comprising an amino acid sequence according to SEQ ID NO:3 (or portion thereof).

In further contemplated aspects, hinge domains of antibodies, such as an IgG, IgA, IgM, IgE, or IgD antibodies, are also deemed suitable for use in the chimeric receptors described herein. In some embodiments, the hinge domain is the hinge domain that joins the constant domains CH1 and CH2 of an antibody. In other embodiments, the hinge domain is of an antibody and comprises the hinge domain of the antibody and one or more constant regions of the antibody. In further embodiments, the hinge domain comprises the hinge domain of an antibody and the CH3 constant region of the antibody. In still further embodiments, the hinge domain comprises the hinge domain of an antibody and the CH2 and CH3 constant regions of the antibody.

In further preferred aspects, the antibody binding domain and the hinge portion (where present) is anchored in the cell membrane via a transmembrane portion. For example, in some embodiments, the transmembrane domain of the chimeric receptor described herein may be derived from a Type I single-pass membrane protein. Single-pass membrane proteins include CD8α, CD8β, 4-1BB/CD137, CD28, CD34, CD4, FcϵRIγ, CD16, OX40/CD134, CD3ζ, CD3ϵ, CD3γ, CD3δ, TCRα, TCRβ, TCζ, CD32, CD64, CD45, CDS, CD9, CD22, CD37, CD80, CD86, CD40, CD4OL/CD154, VEGFR2, FAS, and FGFR2B. In preferred examples, the transmembrane domain is derived from CD8α or CD28 or CD34. Therefore, especially preferred transmembrane portions will have or comprise an amino acid sequence according to SEQ ID NO:5, SEQ ID NO:74, SEQ ID NO:76, SEQ ID NO:78, SEQ ID NO:80, SEQ ID NO:82, or SEQ ID NO:84 (or portion thereof with respect to any of the immediately preceding amino acid sequences).

In still further aspects, the transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, for example, the N-terminus of the protein may be present on the cytoplasmic side of the lipid bilayer and the C-terminus of the protein may be present on the extracellular side. Either one or multiple helix passes from a multi-pass membrane protein can be used for constructing the chimeric receptor described herein.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and WO 2000/032776, both of which are incorporated by reference herein.

With respect to suitable signaling domains it generally contemplated that all signaling portions (typically intracellular) are deemed suitable for use herein, which may include signaling portions of surface receptors such as CD3ζ and FcεRIγ as well as signaling portions of co-stimulatory proteins such as members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6), members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACl/TNFRSF13B, TL1A/TNFSF15, TNF-alpha, and TNF RII/TNFRSF1B), members of the SLAM family (e.g., 2B4/CD244/SLAMF4, BLAME/SLAMF8, CD2, CD2F-10/SLAMF9, CD48/SLAMF2, CD58/LFA-3, CD84/SLAMFS, CD229/SLAMF3, CRACC/SLAMF7, NTB-A/SLAMF6, and SLAM/CD150), and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

However, especially preferred signaling domains include those from CD3ζ having or comprising an amino acid sequence according to SEQ ID NO:9 (or portion thereof), FcεRIγ having or comprising an amino acid sequence according to SEQ ID NO:1 (or portion thereof), CD28 having or comprising an amino acid sequence according to SEQ ID NO:7 (or portion thereof), and 4-1BB having or comprising an amino acid sequence according to SEQ ID NO:8 (or portion thereof).

Therefore, and viewed from yet another perspective, the inventors contemplate various CAR constructs according to Table 1 in which any one of the domains can be built using one or more of the amino acid sequences presented herein. Moreover, while the table below lists exemplary sequences, it should be appreciated that each sequence as identified may include one or more amino acid changes such that the changes amino acid will have an identity of at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 91% to the sequence shown in the table. Moreover, it should also be recognized that the sequences shown in the table may be truncated (at either or both ends) to a shorter sequence so long as the truncated sequence will still retain the indicated function. Of course, it should be recognized that these sequences represent the mature polypeptide sequences without any further sequence portions for export or trafficking (e.g., leader peptide).

TABLE 1

| Functional Domain | Contemplated Polypeptide Sequence |
|---|---|
| Antibody binding domain | SEQ ID NO: 12, 16, 19, 22, 25, 28, 31, |
| | 17, 20, 23, 26, 29, 32 |
| Optional Hinge portion | SEQ ID NO: 3 |
| Transmembrane portion | SEQ ID NO: 5, SEQ ID NO: 74, |
| | SEQ ID NO: 76, SEQ ID NO: 78, |
| | SEQ ID NO: 80, SEQ ID NO: 82, or |
| | SEQ ID NO: 84 |
| Signaling domain | SEQ ID NO: 1, 7, 8, 9 |

In further aspects of the inventive subject matter, it should be recognized that not only the polypeptide sequences presented herein are contemplated, but also nucleic acid sequences and constructs that encode the sequences contemplated herein. Of course, as will be readily appreciated, contemplated nucleic acid sequences may make use of all codon usage patterns, and particularly human codon usage. In addition, the recombinant nucleic acids will also include all required regulatory elements to effect expression of the CAR construct in a cell transfected with the recombinant nucleic acid. A variety of known promoters can be used for expression of the CAR constructs described herein, including the cytomegalovirus (CMV) intermediate early promoter, a viral LTR such as the Rous sarcoma virus LTR, the HIV-LTR, the HTLV-1 LTR, the simian virus 40 (SV40) early promoter, the herpes simplex tk virus promoter, etc. Additional promoters for expression of the chimeric receptors include any constitutively active promoter in an immune cell. Alternatively, any regulatable promoter may be used, such that its expression can be modulated within an immune cell.

Moreover, and where desired, the recombinant nucleic acid may comprise one or more additional sequence portions that may encode one or more additional proteins with a desired function. For example, suitable additional sequence portions will include cytokines, and particularly cytokines required for autocrine growth stimulation of NK cells such as IL-2 and/or IL15, which may be intracellularly retained via an endoplasmic retention sequence, immune stimulatory cytokines such as N-801, interferon gamma, etc., as well as one or more functional proteins that assist in cell migration (e.g., chemokine receptors) or modification of the tumor microenvironment (e.g., IL-8 or TGF-(3 trap).

Additionally, the recombinant nucleic acid may contain further functional elements such as a selectable marker gene (e.g., neomycin gene for selection of stable or transient transfectants in host cells), one or more enhancer/promoter sequences from the immediate early gene of human CMV for increased levels of transcription, a transcription termination and RNA processing signals from SV40 for mRNA stability, SV40 polyoma origins of replication and ColE1 for replication in a bacterium, one or more internal ribosome binding sites (IRESes), multiple cloning sites, T7 and SP6 RNA promoters for in vitro transcription of sense and antisense RNA, a “suicide switch” or “suicide gene” which, when triggered causes cells carrying the vector to die (e.g., HSV thymidine kinase, an inducible caspase such as iCasp9), and/or one or more reporter genes for assessing expression of the CAR construct. Exemplary polycistronic constructs are described in WO 2019/226708, which is incorporated by reference herein. To that end, contemplated nucleic acid constructs may comprise a sequence that encodes a 2A peptide, such as a T2A, P2A, E2A, or F2A peptide, in order to produce equimolar levels of polypeptides encoded by the same mRNA.

Among various other nucleic acid sequences, exemplary nucleic sequences will have a sequence according to SEQ ID NO:2 (encoding Featly intracellular signaling domain), SEQ ID NO:4 (encoding human CD8 hinge), SEQ ID NO:6 (encoding human CD28 transmembrane portion), SEQ ID NO:10 (encoding CD3ζ intracellular signaling domain), SEQ ID NO:11 (encoding low affinity CD16A), SEQ ID NO:13 (encoding high affinity CD16A), SEQ ID NO:15 (encoding CD64A), SEQ ID NO:18 (encoding CD64B), SEQ ID NO:21 (encoding CD64C), SEQ ID NO:24 (encoding CD32A), SEQ ID NO:27 (encoding CD32B), and SEQ ID NO:30 (encoding CD16B). Additional contemplated nucleic acid sequences will have a nucleic acid sequence according to SEQ ID NO:73 (encoding CD16A transmembrane domain), SEQ ID NO:75 (encoding CD32A transmembrane domain), SEQ ID NO:77 (encoding CD32B transmembrane domain), SEQ ID NO:79 (encoding CD64A transmembrane domain), SEQ ID NO:81 (encoding CD64B transmembrane domain), or SEQ ID NO:83 (encoding CD16C transmembrane domain). As noted above, it should be recognized that the nucleic acid sequences as listed above may vary to some degree, for example due to codon preference and one or more amino acid exchanges (preferably while maintaining their respective function). Therefore, nucleic acid sequences contemplated herein also include nucleic acid sequences with a sequence identity of at least 99%, or at least 98%, or at least 97%, or at least 96%, or at least 95%, or at least 94%, or at least 93%, or at least 92%, or at least 91%, or at least 90% to those nucleic acid sequences disclosed herein. Consequently, amino acid sequences contemplated herein also include amino acid sequences with a sequence identity of at least 99%, or at least 98%, or at least 97%, or at least 96%, or at least 95%, or at least 94%, or at least 93%, or at least 92%, or at least 91%, or at least 90% to those amino acid sequences disclosed herein.

Therefore, contemplated nucleic acids sequences will also include various recombinant constructs suitable for transfection, propagation, and/or expression of the CAR construct. For example, such recombinant nucleic acids will include linear or circular DNA and RNA such linearized DNA and RNA, cloning vectors, expression vectors, and even recombinant viruses. Among other preferred options, such constructs will typically be configured as polycistronic constructs in either linearized form, viral form (e.g., adenovirus or lentivirus) or viral expression vector.

In still further contemplated aspects, the CAR constructs presented herein will typically be expressed in a mammalian cell, and most preferably in a therapeutic cell or immune competent cells that can be autologous or heterologous with respect to the individual receiving the cell. For example, suitable cells for expression of the CAR constructs presented herein especially include T cells, NK cells, and NKT cells.

With respect to suitable NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56bright, CD2, CD7, CD11a, CD28, CD45, and CD54 surface markers and did not display the CD1, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-1α, IL-6, tumor necrosis factor α, interferon α, and interferon γ. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407. Still further contemplated NK-92 cells include those that have been genetically engineered to express a cytokine for autocrine growth stimulation, and/or that have been genetically engineered to express a high-affinity version of CD16.

In still further embodiments, the inventors contemplate use of recombinant cells expressing one or more CAR constructs presented herein in the treatment of disease (e.g., cancer, viral infection, bacterial infection, etc.), and especially a disease for which therapeutic antibodies are available. In such treatment uses and methods, a therapeutically effective quantity of recombinant cells will be administered either alone, or in conjunction with a therapeutic antibody.

Contemplated diseases especially include leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Contemplated transfected cells (e.g., transfected NK-92) cells can be administered to an individual by absolute numbers of cells. For example, the individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ cells per injection, or any ranges between any two of the numbers, end points inclusive. In other embodiments, the cells can be administered to an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ cells per kilogram of the individual, or any ranges between any two of the numbers, end points inclusive. In other embodiments, the total dose may calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient.

The transfected cells (e.g., transfected NK-92 cells), and optionally other anti-cancer or anti-viral agents can be administered once to a patient with cancer or infected with a virus or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

Examples

Exemplary nucleic acid and amino acid sequences used in conjunction with the teachings presented herein include SEQ ID NO:33-72. More specifically, SEQ ID NO:33 shows a nucleic acid encoding an exemplary CD16aV CAR with ECD-CD16a TM-FceRIg domains, SEQ ID NO:34 shows an amino acid for an exemplary CD16aV CAR with ECD-CD16a TM-FceRIg domains, SEQ ID NO:35 shows a nucleic acid encoding an exemplary CD16aV CAR with ECD-CD28 TM-FceRIg domains, SEQ ID NO:36 shows an amino acid for an exemplary CD16aV CAR with ECD-CD28 TM-FceRIg domains, SEQ ID NO:37 shows a nucleic acid encoding an exemplary CD16aV CAR with ECD-CD8-CD28 TM -FceRIg domains, SEQ ID NO:38 shows an amino acid for an exemplary CAR comprising CD16aV ECD-CD8-CD28 TM-FceRIg domains, SEQ ID NO:39 shows a nucleic acid encoding an exemplary CAR comprising CD16b ECD-CD8-CD28 TM-FceRIg domains, SEQ ID NO:40 shows an amino acid for an exemplary CAR comprising CD16b ECD-CD8-CD28 TM-FceRIg domains, SEQ ID NO:41 shows a nucleic acid encoding an exemplary CAR comprising CD16b ECD-CD28 TM-FceRIg domains, SEQ ID NO:42 shows an amino acid for an exemplary CAR comprising CD16b ECD-CD28 TM-FceRIg domains, SEQ ID NO:43 shows a nucleic acid encoding an exemplary CAR comprising CD64a ECD-CD64 TM-FceRIg domains, SEQ ID NO:44 shows an amino acid for an exemplary CAR comprising CD64a ECD-CD64 TM-FceRIg domains, SEQ ID NO:45 shows a nucleic acid encoding an exemplary CAR comprising CD64a ECD-CD28 TM-FceRIg domains, SEQ ID NO:46 shows an amino acid for an exemplary CAR comprising CD64a ECD-CD28 TM-FceRIg domains, SEQ ID NO:47 shows a nucleic acid encoding an exemplary CAR comprising CD64a ECD-CD8-CD28 TM-FceRIg domains, SEQ ID NO:48 shows an amino acid for an exemplary CAR comprising CD64a ECD-CD8-CD28 TM-FceRIg domains, SEQ ID NO:49 shows a nucleic acid encoding an exemplary CAR comprising CD64b ECD-CD64 TM-FceRIg domains, SEQ ID NO:50 shows an amino acid for an exemplary CAR comprising CD64b ECD-CD64 TM-FceRIg domains, SEQ ID NO:51 shows a nucleic acid encoding an exemplary CAR comprising CD64b ECD-CD28 TM-FceRIg domains, SEQ ID NO:52 shows an amino acid for an exemplary CAR comprising CD64b ECD-CD28 TM-FceRIg domains, SEQ ID NO:53 shows a nucleic acid encoding an exemplary CAR comprising CD64b ECD-CD8-CD28 TM-FceRIg domains, SEQ ID NO:54 shows an amino acid for an exemplary CAR comprising CD64b ECD-CD8-CD28 TM-FceRIg domains, SEQ ID NO:55 shows a nucleic acid encoding an exemplary CAR comprising CD64c ECD-CD64 TM-FceRIg domains, SEQ ID NO:56 shows an amino acid for an exemplary CAR comprising CD64c ECD-CD64 TM-FceRIg domains, SEQ ID NO:57 shows a nucleic acid encoding an exemplary CAR comprising CD64c ECD-CD28 TM-FceRIg domains, SEQ ID NO:58 shows an amino acid for an exemplary CAR comprising CD64c ECD-CD28 TM-FceRIg domains, SEQ ID NO:59 shows a nucleic acid encoding an exemplary comprising CD64c ECD-CD8-CD28 TM-FceRIg domains, SEQ ID NO:60 shows an amino acid for an exemplary CAR comprising CD64c ECD-CD8-CD28 TM-FceRIg domains, SEQ ID NO:61 shows a nucleic acid encoding an exemplary CAR comprising CD32a ECD-CD32a TM-FceRIg domains, SEQ ID NO:62 shows an amino acid for an exemplary CAR comprising CD32a ECD-CD32a TM-FceRIg domains, SEQ ID NO:63 shows a nucleic acid encoding an exemplary CAR comprising CD32a ECD-CD28 TM-FceRIg domains, SEQ ID NO:64 shows an amino acid for an exemplary CAR comprising CD32a ECD-CD28 TM-FceRIg domains, SEQ ID NO:65 shows a nucleic acid encoding an exemplary CAR comprising CD32a ECD-CD8-CD28 TM-FceRIg domains, SEQ ID NO:66 shows an amino acid for an exemplary CAR comprising CD32a ECD-CD8-CD28 TM-FceRIg domains, SEQ ID NO:67 shows a nucleic acid encoding an exemplary CAR comprising CD32b ECD-CD32b TM-FceRIg domains, SEQ ID NO:68 shows an amino acid for an exemplary CAR comprising CD32b ECD-CD32b TM-FceRIg domains, SEQ ID NO:69 shows a nucleic acid encoding an exemplary CAR comprising CD32b ECD-CD28 TM-FceRIg domains, SEQ ID NO:70 shows an amino acid for an exemplary CAR comprising CD32b ECD-CD28 TM-FceRIg domains, SEQ ID NO:71 shows a nucleic acid encoding an exemplary CAR comprising CD32b ECD-CD8-CD28 TM-FceRIg domains, and SEQ ID NO:72 shows an amino acid for an exemplary CAR comprising CD32b ECD-CD8-CD28 TM-FceRIg domains.

Here ECD refers to the extracellular domains of an Fc receptor (which, for example, may be qualified as ‘CD32b ECD’ for the extracellular domain of the CD32B Fc receptor, or as ECD-CD16a for the extracellular domain of the CD16a Fc receptor), TM refers to a transmembrane domain (e.g., CD28 transmembrane domain CD28 TM), FceRIg refers to the signaling domain form FceRIg, and CD8 refers to a CD8 hinge domain.

Further exemplary nucleic acid and amino acid sequences used in conjunction with the teachings presented herein include SEQ ID NO:73-84. More specifically, SEQ ID NO:73 shows a nucleic acid encoding an exemplary CD16A transmembrane domain, SEQ ID NO:74 shows an amino acid for an exemplary CD16A transmembrane domain, SEQ ID NO:75 shows a nucleic acid encoding an exemplary CD32A transmembrane domain, SEQ ID NO:76 shows an amino acid for an exemplary CD32A transmembrane domain, SEQ ID NO:77 shows a nucleic acid encoding an exemplary CD32B transmembrane domain, SEQ ID NO:78 shows an amino acid for an exemplary CD32B transmembrane domain, SEQ ID NO:79 shows a nucleic acid encoding an exemplary CD64A transmembrane domain, SEQ ID NO:80 shows an amino acid for an exemplary CD64A transmembrane domain, SEQ ID NO:81 shows a nucleic acid encoding an exemplary CD64B transmembrane domain, SEQ ID NO:82 shows an amino acid for an exemplary CD64B transmembrane domain, SEQ ID NO:83 shows a nucleic acid encoding an exemplary CD64C transmembrane domain, and SEQ ID NO:84 shows an amino acid for an exemplary CD64C transmembrane domain. In this context, it should be appreciated that all of the transmembrane domain sequences can be used in the CAR constructs as described herein interchangeably. Therefore, and for example, an exemplary CAR comprising CD32b ECD-CD8-CD28 TM-FceRIg domains can also be prepared as a CAR that includes instead of the CD28 TM domain any one of the CD16A, CD32A, CD32B, CD64A, CD64B, or CD64C TM domains as shown above in SEQ ID NOs: 73-84.

Example 1. Transfection of aNK cells: Fc-CAR aNK cells are generated by electroporating aNK cells with a bicistronic plasmid-based vector containing sequences for Fc-CAR and IL-2. The IL-2 sequence is tagged with the endoplasmic reticulum retention signal, KDEL, to prevent IL-2 protein secretion from the endoplasmic reticulum (ER), and is referred to as ERIL-2.

The nucleic acids encoding the various Fc-CAR constructs are provided in the sequence listing and may or may not include a hinge region. These constructs will be assembled from synthetic oligonucleotides and PCR products generated by GeneWiz, Inc. The constructs will be cloned into a bicistronic pNEUKv1 IRES_ERIL2 vector backbone, containing an ampicillin resistance cassette, the EF-1 alpha promoter, and an SV40 polyadenylation sequence. The resulting plasmid DNAs will be purified from transformed bacteria and their concentration will be determined by UV spectroscopy. aNK cells will be electroporated with the various purified Fc-CAR plasmids using a Neon electroporator device. Electroporated cells will be put back into X-VIVO 10 medium supplemented with 5% heat-inactivated human AB serum, without addition of IL-2, and incubated at 37° C. in a 5% CO₂ incubator.

Expected results: Polyclonal aNK cell populations that successfully incorporated the plasmid constructs will able to grow in absence of IL-2 in the culture medium. An expanding population of Fc-CAR aNK cells should be detectable within 3 to 5 weeks of electroporation, and amenable to testing within another 2 to 3 weeks.

Example 2. Phenotyping: Flow cytometry analysis will be conducted to measure the surface expression of the Fc-CAR on the electroporated aNK cells. Cells will be stained with fluorochrome-conjugated antibodies recognizing human CD16, CD32, or CD64 according to the manufacturer's instructions and analyzed on a flow cytometer device.

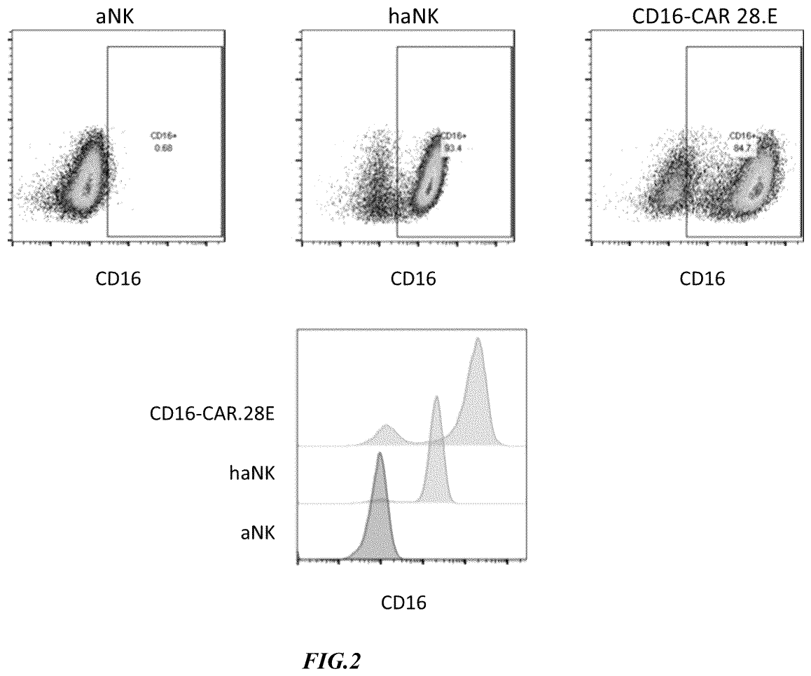

Expected results: Surface expression of Fc-CAR molecules should be observed in at least 30% of the cells after 3 to 5 weeks culture following electroporation. More specifically, in a selected experimental result, FIG. 2 depicts the data for a CD16-CAR.28E construct expressed in aNK cells (CD16-CAR.28E cells), in which the CAR was constructed from the extracellular domain of CD16A-158V variant of FCGRIIIA, the transmembrane domain of CD28, and the signaling domain of FCERIG. The nucleic acid encoding such construct was subcloned into the pNEUKv1-IRES-ERIL2 plasmid. aNK cells were electroporated with this plasmid using a Neon™ electroporator device. Three weeks after electroporation, cells were stained with an anti-CD16 antibody and analyzed by flow cytometry. Electroporated cells were compared to non-electroporated aNK and haNK cells. As can be readily seen from the scans, aNK cells did not express CD16 on their cell surface, whereas haNK cells showed significant CD16 expression on the cell surface. Likewise, CD16-CAR 28.E cells had a strong signal for CD16 surface presentation.

Example 3. Direct or natural cytotoxicity: K562 cells will be grown in RPMI-1640 medium (Gibco/Thermofisher) supplemented with 10% heat-inactivated FBS (Gibco/Thermofisher) and incubated at 37° C. in a 5% CO₂ incubator. K562 cells will be stained with a green fluorescent dye (PKH67-GL), and Fc-CAR aNK effector cells will be combined at different effector to target (E:T) ratio in a 96-well plate, briefly centrifuged, and incubated at 37° C. for 4 h in a 5% CO₂ incubator. After incubation, cells will be stained with propidium iodide (PI) at 1 μg/ml in 1% BSA/PBS buffer and analyzed immediately by flow cytometry. Target cells and effector cells will also be stained separately with PI to assess spontaneous cell lysis.

Dead target cells will be identified as double positive for PKH67-GL and PI. Percentage of dead cells will be determined by the percentage of PI⁺ within the PKH67⁺ target cell population. % Killing will be calculated as follows=[% dead target cells in sample−% spontaneous dead target cells]/[100−% spontaneous dead target cells].

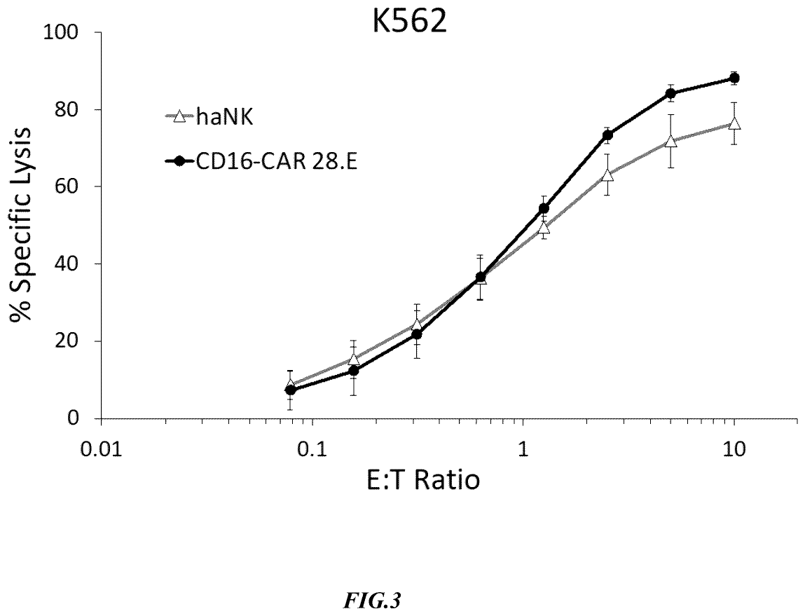

Expected results: Percentage killing of K562 target cells should be over 50% at the highest E:T ratio. More specifically, in a selected experimental result, FIG. 3 depicts the data for cytotoxic activity of CD16-CAR.28E cells as determined by a flow based in vitro cytotoxicity assay against the NK-sensitive human cell line K562 and compared to that of haNK cells. Effector and target cells were mixed a E:T ratios ranging from 10:1 to 0.06:1 and incubated at 37° C. for 4 hours. As can be readily taken from the graph in FIG. 3, cytotoxicity was comparable, and at higher E:T ratios even better than cytotoxicity of haNK cells.

Example 4. ADCC: A CD20-expressing variant of the NK-resistant SUP-B15 target cells will be used for the assay. CD20+SUP-B15 target cells will be grown in RPMI-1640 medium (Gibco/Thermofisher) supplemented with 20% heat-inactivated FBS (Gibco/Thermofisher) and 0.2% beta-mercaptoethanol and incubated at 37° C. in a 5% CO2 incubator. CD20+SUP-B15 cells will be stained with a green-fluorescent dye (PKH67-GL). Stained target cells will be then pre-incubated with the monoclonal antibodies rituximab (anti-CD20 antibody) or trastuzumab (anti-HER2/neu control antibody, SUP-B15 cells being HER2/neu negative) at a concentration of 2 μg/ml for 20 minutes, or without antibody. Pre-incubated stained target cells will be combined with Fc-CAR aNK effector cells at different effector to target (E:T) ratios in a 96-well plate, briefly centrifuged, and incubated at 37° C. for 4 h in a 5% CO2 incubator. After incubation, cells will be stained with propidium iodide (PI) at 1 μg/ml in 1% BSA/PBS buffer and analyzed immediately by flow cytometry. Target cells and effector cells will be stained separately with PI to assess spontaneous cell lysis. Dead target cells are identified as double positive for PKH67-GL and PI. The antibody-dependent cell-mediated cytotoxicity (% ADCC) will be calculated as follows=[(% dead target cells in sample E+T plus mAb)−(% dead target cells in sample E+T minus mAb)]/[100−(% dead target cells in sample E+T minus mAb)], (E=effector, T=target, mAb=monoclonal antibody).

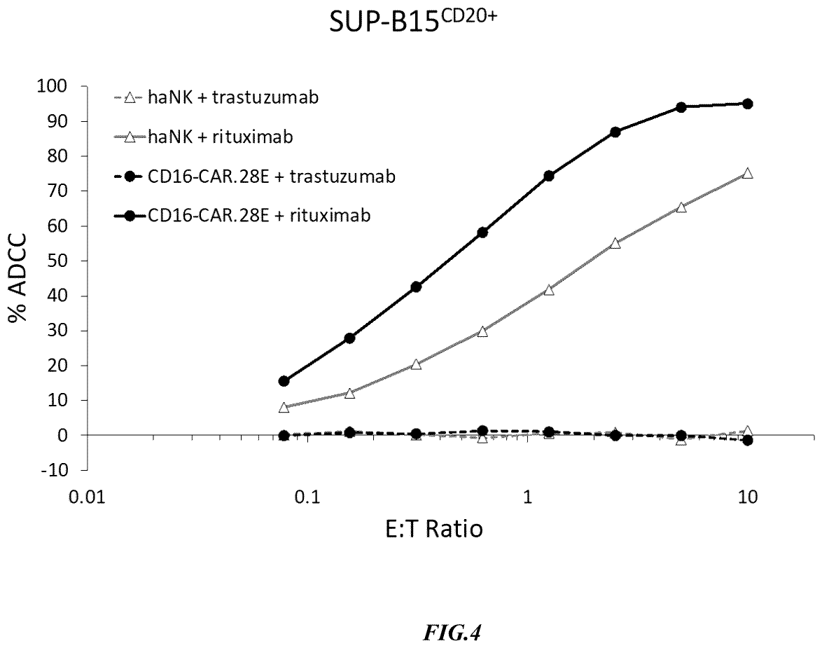

Expected results: Percentage ADCC killing of CD20+SUP-B15 target cells should be significantly higher in presence of rituximab than in presence of trastuzumab. % ADCC should be at least 20% at the highest E:T ratio. More specifically, in a selected experimental result, FIG. 4 depicts the data for antibody-dependent cell-mediated cytotoxic (ADCC) activity of CD16-CAR.28E cells as determined by a flow based in vitro cytotoxicity assay against the CD20-positive, HER2-negative, NK-resistant human cell line SUP-B15^{CD20+}. Target cells were pre-incubated for 20 min at room temperature with either rituximab (on-target anti-CD20) or trastuzumab (off-target anti-HER2) antibodies at 2 μg/mL, or without antibody. Effector and pre-incubated target cells were then mixed a E:T ratios ranging from 10:1 to 0.06:1 and incubated at 37oC for 4 hours. haNK cells were included in the assay for comparison. As can be clearly seen form the graph in FIG. 4, both haNK cells and CD16-CAR.28E cells had no ADCC activity in the presence of off-target antibodies. Conversely, both haNK cells and CD16-CAR.28E cells had significant ADCC activity in the presence of on-target antibodies, with the CD16-CAR.28E cells significantly outperforming haNK cells. Viewed from a different perspective, the CD16-CAR.28E construct unexpectedly provided stronger ADCC activity than a comparable NK cell expressing the CD16 158V high affinity variant on the cell surface.

Further aspects, considerations, and methods suitable for use in conjunction with the teachings presented herein are described in US 2018/0133252 and US 2016/0067356, both of which are incorporated by reference herein.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification and/or claims refer to at least one of something selected from the group consisting of A, B, C . . . . and N, the text should be interpreted as requiring only one element from the group, not A plus N, or B plus N, etc.

What is claimed is:

1. A recombinant chimeric antigen receptor (CAR), comprising:
an antibody binding domain having an antibody-binding portion of a polypeptide with a sequence selected from the group consisting of SEQ ID NO:12, SEQ ID NO:16, SEQ ID NO:19, SEQ ID NO:22, SEQ ID NO:25, SEQ ID NO:28, and SEQ ID NO:31; and
wherein the antibody binding domain is coupled to a polypeptide comprising in sequence an optional hinge portion, a transmembrane portion, and a signaling domain.

2. The chimeric antigen receptor of claim 1 wherein the antibody binding domain has a peptide sequence selected from the group consisting of SEQ ID NO:17, SEQ ID NO:20, SEQ ID NO:23, SEQ ID NO:26, SEQ ID NO:29, and SEQ ID NO:32.

3. The chimeric antigen receptor of any one of the preceding claims wherein the optional hinge portion has a peptide sequence of SEQ ID NO:3.

4. The chimeric antigen receptor of any one of the preceding claims wherein the transmembrane portion has a peptide sequence of SEQ ID NO:5, SEQ ID NO:74, SEQ ID NO:76, SEQ ID NO:78, SEQ ID NO:80, SEQ ID NO:82, or SEQ ID NO:84.

5. The chimeric antigen receptor of any one of the preceding claims wherein the signaling domain has a peptide sequence of SEQ ID NO:1.

6. The chimeric antigen receptor of any one of the preceding claims further comprising at least one second signaling domain.

7. The chimeric antigen receptor of claim 6 wherein the second signaling domain is distinct from the signaling domain.

8. The chimeric antigen receptor of any one of the preceding claims wherein the signaling domain has a peptide sequence selected from the group consisting of SEQ ID NO:7, SEQ ID NO:8, and SEQ ID NO:9.

9. The chimeric antigen receptor of claim 1 wherein the antibody binding domain has a peptide sequence selected from the group consisting of SEQ ID NO:17, SEQ ID NO:20, SEQ ID NO:23, SEQ ID NO:26, SEQ ID NO:29, and SEQ ID NO:32, and wherein the signaling domain has a peptide sequence of SEQ ID NO:1.

10. The chimeric antigen receptor of claim 9 wherein the optional hinge portion has a peptide sequence of SEQ ID NO:3.

11. The chimeric antigen receptor of claim 9 or claim 10 wherein the transmembrane portion has a peptide sequence of SEQ ID NO:5, SEQ ID NO:74, SEQ ID NO:76, SEQ ID NO:78, SEQ ID NO:80, SEQ ID NO:82, or SEQ ID NO:84.

12. The chimeric antigen receptor of any one of claims 9-11 further comprising at least one additional signaling domain, and optionally wherein the additional signaling domain has a peptide sequence of SEQ ID NO:1.

13. The chimeric antigen receptor of any one of claims 9-12 wherein the additional signaling domain has a peptide sequence of SEQ ID NO:1.

14. The chimeric antigen receptor of claim 1 wherein the chimeric antigen receptor has an amino acid sequence of SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54xx, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or SEQ ID NO:72.

15. The chimeric antigen receptor of claim 1 wherein the chimeric antigen receptor is encoded by a nucleic acid having a nucleotide sequence of SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71.

16. A recombinant nucleic acid encoding the chimeric antigen receptor of any one of claims 1-13.

17. The recombinant nucleic acid of claim 16 wherein the nucleic acid is codon-optimized to human codon usage.

18. The recombinant nucleic acid of any one of claims 16-17, further comprising a sequence portion that encodes a cytokine, a CD16, a homing receptor, and/or a TGF-beta trap.

19. The recombinant nucleic acid of claim 18 wherein the nucleic acid encodes the chimeric antigen receptor and the sequence portion that encodes the cytokine, the CD16, the homing receptor, and/or the TGF-beta trap is configured as a polycistronic nucleic acid.

20. The recombinant nucleic acid of any one of claims 16-19, wherein the recombinant nucleic acid is part of a lentiviral vector.

21. The recombinant nucleic acid of any one of claims 16-19, wherein the recombinant nucleic acid is part of a DNA vector.

22. A cell transfected with the recombinant nucleic acid of any one of claims 16-21.

23. The cell of claim 22 wherein the cell is a NK cell or a T cell.

24. The cell of claim 22 wherein the cell is an NK-92 cell, a genetically modified NK-92 cell, or an autologous NK cell.

25. A recombinant NK cell that is transfected with a recombinant nucleic acid encoding a recombinant chimeric antigen receptor of any one of claims 1-13 or claim 15.

26. The recombinant NK cell of claim 25 wherein the NK cell is an NK-92 cell, a genetically modified NK-92 cell, or an autologous NK cell.

27. The recombinant NK cell of claim 25 transfected with the recombinant nucleic acid of any one of claims 14-19.

28. A method of treating cancer in a patient in need thereof, comprising administering to the patient a therapeutically effective amount of the cells of any one of claims 22-27, thereby treating the cancer.

29. The method of claim 28 further comprising a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine.

30. The method of claim 28 or 29, wherein the cancer is selected from leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

31. The method of any one of claims 28-30, wherein about 1×10⁸ to about 1×10¹¹ cells per m² of body surface area of the patient are administered to the patient.

32. Use of a cell of any one of claims 22-27 in the treatment of cancer or a viral infection.


SEQUENCE LISTING (84 sequences)

SEQ ID NO: 1 - protein, 41 aa, Homo sapiens
  Misc_feature (1)..(41): FceRIgamma Intracellular Domain peptide
LKIQVRKAAITSYEKSDGVYTGLSTRNQETYETLKHEKPPQ

SEQ ID NO: 2 - DNA, 123 bp, Homo sapiens
  Misc_feature (1)..(123): FceRIgamma Intracellular Domain
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 3 - protein, 64 aa, Homo sapiens
  Misc_feature (1)..(64): CD8 Hinge (Human)
LSNSIMYFSHFVPVFLPAKPTTTPAPRPPTPAPTIASQPLSLRPEACRPAAGGAVHTRGLDFAC

SEQ ID NO: 4 - DNA, 192 bp, Homo sapiens
  Misc_feature (1)..(192): CD8 Hinge DNA (Human)
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgc

SEQ ID NO: 5 - protein, 28 aa, Homo sapiens
  Misc_feature (1)..(28): Human CD28 Transmembrane domain
FWVLVVVGGVLACYSLLVTVAFIIFWVR

SEQ ID NO: 6 - DNA, 84 bp, Homo sapiens
  Misc_feature (1)..(84): Human CD28 Transmembrane domain (nt)
ttttgggtgctggtggtcgtgggcggagtgctggcttgttattctctgctggtcaccgtggccttcatcatcttttgggtccga

SEQ ID NO: 7 - protein, 41 aa, Homo sapiens
  Misc_feature (1)..(41): CD28 signaling domain
RSKRSRLLHSDYMNMTPRRPGPTRKHYQPYAPPRDFAAYRS

SEQ ID NO: 8 - protein, 42 aa, Homo sapiens
  Misc_feature (1)..(42): 4-1BB signaling domain
KRGRKKLLYIFKQPFMRPVQTTQEEDGCSCRFPEEEEGGCEL

SEQ ID NO: 9 - protein, 113 aa, Homo sapiens
  Misc_feature (1)..(113): CD3 zeta signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - DNA, 339 bp, Homo sapiens
  Misc_feature (1)..(339): Human CD3 zeta
gtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 11 - DNA, 765 bp, Homo sapiens
  MIsc_feature (1)..(765): Polynucleotide Encoding the Low Affinity Immunoglobulin Gamma Fc Region Receptor III-A
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 12 - protein, 254 aa, Homo sapiens
  Misc_feature (1)..(254): High Affinity Variant Ig gamma FcRIII-A amino acid sequence
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 13 - DNA, 765 bp, Homo sapiens
  Misc_feature (1)..(765): High Affinity Variant IgGamma FcRIII-A nucleic acid sequence
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 14 - protein, 135 aa, Homo sapiens
  Misc_feature (1)..(135): Human T-cell surface glycoprotein CD3 zeta
PKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1336 bp, Homo sapiens
  Misc_feature (1)..(1336): Human FCGR1A (CD64A) nt sequence
aatatcttgcatgttacagatttcactgctcccaccagcttggagacaacatgtggttcttgacaactctgctcctttgggttccagttgatgggcaagtggtagacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatctggaccgtcccctgcccacttgctccccgtgagcactgcgtacaaacatccaaaagttcaacaacaccagaactgtgtgtctcatggtatgtaactcttaaagcaaataaatgaactgacttcaactggga

SEQ ID NO: 16 - protein, 374 aa, Homo sapiens
  Misc_feature (1)..(374): Human FCGR1A (CD64A) a.a. sequence
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 17 - protein, 292 aa, Homo sapiens
  Misc_feature (1)..(292): Human FCGR1A (CD64A) extracellular domain
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFH

SEQ ID NO: 18 - DNA, 904 bp, Homo sapiens
  Misc_feature (1)..(904): Human FCGR1B (CD64B)
gtacaaaaaagcagaagggccgtcaaggcccaccatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtaaggcctcatgggcccagctttcttgtac

SEQ ID NO: 19 - protein, 280 aa, Homo sapiens
  Misc_feature (1)..(280): Human FCGR1B (CD64B)
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 20 - protein, 198 aa, Homo sapiens
  Misc_feature (1)..(198): Human FCGR1B (CD64B) extracellular domain
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFH

SEQ ID NO: 21 - DNA, 1068 bp, Homo sapiens
  Misc_feature (1)..(1352): Human FCGR1C
aatatcttgcatgttacagatttcactgctcccaccagcttggagacaacatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggccactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcaagggaaagcatcactacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatctggaccgtcccctgtccacttgctccccgtgagcactgcgtacaaacatccaaaagttcaacaacaccagaactgtgtgtctcatggtatataactcttaaggcaaataaatgaactgacttcaactgggaaaaaaaaaaaaaaaaaa

SEQ ID NO: 22 - protein, 280 aa, Homo sapiens
  Misc_feature (1)..(280): Human FCGR1C
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWPLLQVSSRVFTEGEPLALRCHAWKDKLVYNVLYYRNGKAFKFFHWNSNLTILKTNISHNGTYHCSGKGKHHYTSAGISQYTVKGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 23 - protein, 198 aa, Homo sapiens
  Misc_feature (1)..(198): Human FCGR1C extracellular domain
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWPLLQVSSRVFTEGEPLALRCHAWKDKLVYNVLYYRNGKAFKFFHWNSNLTILKTNISHNGTYHCSGKGKHHYTSAGISQYTVKGLQLPTPVWFH

SEQ ID NO: 24 - DNA, 2643 bp, Homo sapiens
  Misc_feature (1)..(2643): Human FCGR2A (CD32A)
acagtgctgggatgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaaagagtaacgttatgccatgtggtcatactctcagcttgctgagtggatgacaaaaagaggggaattgttaaaggaaaatttaaatggagactggaaaaatcctgagcaaacaaaaccacctggcccttagaaatagctttaactttgcttaaactacaaacacaagcaaaacttcacggggtcatactacatacaagcataagcaaaacttaacttggatcatttctggtaaatgcttatgttagaaataagacaaccccagccaatcacaagcagcctactaacatataattaggtgactagggactttctaagaagatacctacccccaaaaaacaattatgtaattgaaaaccaaccgattgcctttattttgcttccacattttcccaataaatacttgcctgtgacattttgccactggaacactaaacttcatgaattgcgcctcagatttttcctttaacatcttttttttttttgacagagtctcaatctgttacccaggctggagtgcagtggtgctatcttggctcactgcaaacccgcctcccaggtttaagcgattctcatgcctcagcctcccagtagctgggattagaggcatgtgccatcatacccagctaatttttgtattttttattttttttttttagtagagacagggtttcgcaatgttggccaggccgatctcgaacttctggcctctagcgatctgcccgcctcggcctcccaaagtgctgggatgaccagcatcagccccaatgtccagcctctttaacatcttctttcctatgccctctctgtggatccctactgctggtttctgccttctccatgctgagaacaaaatcacctattcactgcttatgcagtcggaagctccagaagaacaaagagcccaattaccagaaccacattaagtctccattgttttgccttgggatttgagaagagaattagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgagaaacgcttatgttacaggttacatgagagcaatcatgtaagtctatatgacttcagaaatgttaaaatagactaacctctaacaacaaattaaaagtgattgtttcaaggtgatgcaattattgatgacctattttatttttctataatgatcatatattacctttgtaataaaacattataaccaaaacattctgtttaccttttcagggctgtattgattggggtgtagactgaactatccggggtctgtttcttttcggtgatgaaagtcttgagaaggtagtaatggataagatgtgagggagaggagagagggagatttggagtgtagggtgagtgcccctcttcttagaactgaatactcttcttctaatgaacttgtattcttgtttccatgtcttcttccctttccttctatagcaaataaagcattcactttgttttggaa

SEQ ID NO: 25 - protein, 316 aa, Homo sapiens
  Misc_feature (1)..(316): Human FCGR2A (CD32A)
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 26 - protein, 216 aa, Homo sapiens
  Misc_feature (1)..(216): Human FCGR2A (CD32A) extracellular domain
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMG

SEQ ID NO: 27 - DNA, 2068 bp, Homo sapiens
  Misc_feature (1)..(2068): Human FCGR2B (CD32B)
gcagaccaagtggcttaagggtagaatccgccaagctttgagagaaggctgtgactgctgtgctctgggcgccagctcgctccagggagtgatgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttagtctccattgtcttgcattgggatttgagaagaaaatcagagagggaagatctggtatttcctggcctaaattccccttggggaggacagggagatgctgcagttccaaaagagaaggtttcttccagagtcatctacctgagtcctgaagctccctgtcctgaaagccacagacaatatggtcccaaataaccgactgcaccttctgtgcttcagctcttcttgacatcaaggctcttccgttccacatccacacagccaatccaattaatcaaaccactgttattaacagataatagcaacttgggaaatgcttatgttacaggttacgtgagaacaatcatgtaaatctatatgatttcagaaatgttaaaatagactaacctctaccagcacattaaaagtgattgtttctgggtgataaaattattgatgatttttattttctttatttttctataaagatcatatattacttttataataaaacattataaaaacaacattctgtttaccttttcaaggctgtattggttggagtgtagactgaactgcctggggtctgtttctcttcagtgatgagactcttaggaaggcaggaatggataggatagggggaggagaggagagatggggatttagaatgtagagtgagtgccccttttcttaaaactgaatacagtcacgcaccacataatgatgtttagttcaacaacagactgcatatatgatggtgatcccataaaattataataccatatttctattgtaccttttctattcctatgtttagatatatgagtacttaccattgtgttacaattgcctaaagtattcagtacagtagcatgctgtacaggtttgtagcctaggggcaataggctatacgctacagcctaggtgtgtagtaggccacaccatttaggtttgtataagtacctgctatgatgttcacacaacaaaattgcctgcatttctcaaaatgtatccccatatttcaacaatgcatgactgtactcttctgccaatgaccttgtattcttgtttccatgtcttcttctctttcctcctatggcaaataaaacactgttttgcaacacaa

SEQ ID NO: 28 - protein, 310 aa, Homo sapiens
  Misc_feature (1)..(310): Human FCGR2B (CD32B)
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGTAVAAIVAAVVALIYCRKKRISALPGYPECREMGETLPEKPANPTNPDEADKVGAENTITYSLLMHPDALEEPDDQNRI

SEQ ID NO: 29 - protein, 217 aa, Homo sapiens
  Misc_feature (1)..(217): Human FCGR2B (CD32B) extracellular domain
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAP

SEQ ID NO: 30 - DNA, 2300 bp, Homo sapiens
  Misc_feature (1)..(2300): Human FCGR3B (CD16B)
agatgctcagccgcagacctttgggagagtaaagggggcacacccacccaccttgcctccaggctctttccttcctattcctgttctatggtggggctccattgcgagacttcagattgagaaatcagatgaagtttcaagaaaaggaaactggcaggtgacagagatgggtggagggactggggaaaggctgtttactccctcctgtctagtcggcttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagcaaagcaacaatacaggctagggatggtaatcctttaaacatacaaaaattgctcgtattataaattacccagtttagagggaaaaaaagaaaataattattcctaaacaaatggataagtagaattaatggttgaggcaggaccctacagagtgtgggaactgctggggatctagagaattcagtgggaccaatgaaagcatggctgagaaatagcagggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcatgtcagttccaatgaggtggggatggagaagacaattgttgcttatgaaagaaagctttagctgtctctgttttgtaagctttcagtgcaacatttcttggttccaataaagcattttacaagatcttgcatgctactcttagatagaagatggcaaaaccatggtaataaaatatgaatgataaaaaaaaa

SEQ ID NO: 31 - protein, 233 aa, Homo sapiens
  Misc_feature (1)..(233): Human FCGR3B (CD16B)
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 32 - protein, 200 aa, Homo sapiens
  Misc_feature (1)..(200): Human FCGR3B (CD16B) extracellular domain
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTIS

SEQ ID NO: 33 - DNA, 810 bp, Artificial Sequence
  CD16aV CAR with ECD - CD16a TM - FceRIg portions
  misc_feature (1)..(810): CD16aV CAR with ECD - CD16a TM - FceRIg domains
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 34 - protein, 270 aa, Artificial Sequence
  CD16aV CAR with ECD - CD16a TM - FceRIg domains
  MISC_FEATURE (1)..(270): CD16aV CAR with ECD - CD16a TM - FceRIg domains
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVLKIQVRKAAITSYEKSDGVYTGLSTRNQETYETLKHEKPPQ

SEQ ID NO: 35 - DNA, 825 bp, Artificial Sequence
  CD16aV CAR with ECD - CD28 TM - FceRIg domains
  misc_feature (1)..(825): CD16aV CAR with ECD - CD28 TM - FceRIg domains
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 36 - protein, 275 aa, Artificial Sequence
  CD16aV CAR with ECD - CD28 TM - FceRIg domains
  MISC_FEATURE (1)..(275): CD16aV CAR with ECD - CD28 TM - FceRIg domains
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGFWVLVVVGGVLACYSLLVTVAFIIFWVRLKIQVRKAAITSYEKSDGVYTGLSTRNQETYETLKHEKPPQ

SEQ ID NO: 37 - DNA, 1017 bp, Artificial Sequence
  CD16aV CAR with ECD - CD8 - CD28 TM - FceRIg domains
  misc_feature (1)..(1017): CD16aV CAR with ECD - CD8 - CD28 TM - FceRIg domains
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggcctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 38 - protein, 339 aa, Artificial Sequence
  CAR comprising CD16aV ECD - CD8 - CD28 TM - FceRIg domains
  MISC_FEATURE (1)..(339): CAR comprising ECD - CD8 - CD28 TM - FceRIg domains
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 39 - DNA, 999 bp, Artificial Sequence
  CAR comprising CD16b ECD - CD8 - CD28 TM - FceRIg domains
  misc_feature (1)..(998): CAR comprising CD16b ECD - CD8 - CD28 TM - FceRIg domains
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcactgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 40 - protein, 333 aa, Artificial Sequence
  CAR comprising CD16b ECD - CD8 - CD28 TM - FceRIg domains
  MISC_FEATURE (1)..(333): CAR comprising CD16b ECD - CD8 - CD28 TM - FceRIg domains
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 41 - DNA, 807 bp, Artificial Sequence
  CAR comprising CD16b ECD - CD28 TM - FceRIg domains
  misc_feature (1)..(807): CAR comprising CD16b ECD - CD28 TM - FceRIg domians
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcattttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 42 - protein, 269 aa, Artificial Sequence
  CAR comprising CD16b ECD - CD28 TM - FceRIg domains
  MISC_FEATURE (1)..(269): CAR comprising CD16b ECD - CD28 TM - FceRIg domains
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISFWVLVVVGGVLACYSLLVTVAFIIFWVRLKIQVRKAAITSYEKSDGVYTGLSTRNQETYETLKHEKPPQ

SEQ ID NO: 43 - DNA, 1056 bp, Artificial Sequence
  CAR comprising CD64a ECD - CD64 TM - FceRIg domains
  misc_feature (1)..(1056): CAR comprising CD64a ECD - CD64 TM - FceRIg domains
atgtggttcttgacaactctgctcctttgggttccagttgatgggcaagtggtagacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctggctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 44 - protein, 352 aa, Artificial Sequence
  CAR comprising CD64a ECD - CD64 TM - FceRIg domains
  MISC_FEATURE (1)..(352): CAR comprising CD64a ECD - CD64 TM - FceRIg domains
MWFLTTLLLWVPVDGQVVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWLKIQVRKAAITSYEKSDGVYTGLSTRNQETYETLKHEKPPQ

SEQ ID NO: 45 - DNA, 1086 bp, Artificial Sequence
  CAR comprising CD64a ECD - CD28 TM - FceRIg domains
  misc_feature (1)..(1086): CAR comprising CD64a ECD - CD28 TM - FceRIg domains
atgtggttcttgacaactctgctcctttgggttccagttgatgggcaagtggtagacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 46 - protein, 362 aa, Artificial Sequence
  CAR comprising CD64a ECD - CD28 TM - FceRIg domains
  MISC_FEATURE (1)..(362): CAR comprising CD64a ECD - CD28 TM - FceRIg domains
MWFLTTLLLWVPVDGQVVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHFWVLVVVGGVLACYSLLVTVAFIIFWVRLKIQVRKAAITSYEKSDGVYTGLSTRNQETYETLKHEKPPQ

SEQ ID NO: 47 - DNA, 1278 bp, Artificial Sequence
  CAR comprising CD64a ECD - CD8 - CD28 TM - FceRIg domains
  misc_feature (1)..(1278): CAR comprising CD64a ECD - CD8 - CD28 TM - FceRIg domains
atgtggttcttgacaactctgctcctttgggttccagttgatgggcaagtggtagacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 48 - protein, 426 aa, Artificial Sequence
  CAR comprising CD64a ECD - CD8 - CD28 TM - FceRIg domains
MWFLTTLLLWVPVDGQVVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 49 - DNA, 780 bp, Artificial Sequence
  CAR comprising CD64b ECD - CD64 TM - FceRIg domains
  misc_feature (1)..(780): CAR comprising CD64b ECD - CD64 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 50 - protein, 260 aa, Artificial Sequence
  CAR comprising CD64b ECD - CD64 TM - FceRIg domains
  MISC_FEATURE (1)..(260): CAR comprising CD64b ECD - CD64 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHVLFYLAVGIMFLVNTVLWVTILKIQVRKAAITSYEKSDGVYTGLSTRNQETYETLKHEKPPQ

SEQ ID NO: 51 - DNA, 801 bp, Artificial Sequence
  CAR comprising CD64b ECD - CD28 TM - FceRIg domains
  misc_feature (1)..(801): CAR comprising CD64b ECD - CD28 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 52 - protein, 267 aa, Artificial Sequence
  CAR comprising CD64b ECD - CD28 TM - FceRIg domains
  MISC_FEATURE (1)..(267): CAR comprising CD64b ECD - CD28 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHFWVLVVVGGVLACYSLLVTVAFIIFWVRLKIQVRKAAITSYEKSDGVYTGLSTRNQETYETLKHEKPPQ

SEQ ID NO: 53 - DNA, 993 bp, Artificial Sequence
  CAR comprising CD64b ECD - CD8 - CD28 TM - FceRIg domains
  misc_feature (1)..(993): CAR comprising CD64b ECD - CD8 - CD28 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 54 - protein, 331 aa, Artificial Sequence
  CAR comprising CD64b ECD - CD8 - CD28 TM - FceRIg domains
  MISC_FEATURE (1)..(331): CAR comprising CD64b ECD - CD8 - CD28 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 55 - DNA, 771 bp, Artificial Sequence
  CAR comprising CD64c ECD - CD64 TM - FceRIg domains
  misc_feature (1)..(771): CAR comprising CD64c ECD - CD64 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggccactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcaagggaaagcatcactacacatcagcaggaatatcacaatacactgtgaaaggcctccagttaccaactcctgtctggtttcatgtccttttctatctggcagtgggaataatgtttttagtgaacactgttctctggctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 56 - protein, 257 aa, Artificial Sequence
  CAR comprising CD64c ECD - CD64 TM - FceRIg domains
  MISC_FEATURE (1)..(257): CAR comprising CD64c ECD - CD64 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWPLLQVSSRVFTEGEPLALRCHAWKDKLVYNVLYYRNGKAFKFFHWNSNLTILKTNISHNGTYHCSGKGKHHYTSAGISQYTVKGLQLPTPVWFHVLFYLAVGIMFLVNTVLWLKIQVRKAAITSYEKSDGVYTGLSTRNQETYETLKHEKPPQ

SEQ ID NO: 57 - DNA, 801 bp, Artificial Sequence
  CAR comprising CD64c ECD - CD28 TM - FceRIg domains
  misc_feature (1)..(801): CAR comprising CD64c ECD - CD28 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggccactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcaagggaaagcatcactacacatcagcaggaatatcacaatacactgtgaaaggcctccagttaccaactcctgtctggtttcatttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 58 - protein, 267 aa, Artificial Sequence
  CAR comprising CD64c ECD - CD28 TM - FceRIg domains
  MISC_FEATURE (1)..(367): CAR comprising CD64c ECD - CD28 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWPLLQVSSRVFTEGEPLALRCHAWKDKLVYNVLYYRNGKAFKFFHWNSNLTILKTNISHNGTYHCSGKGKHHYTSAGISQYTVKGLQLPTPVWFHFWVLVVVGGVLACYSLLVTVAFIIFWVRLKIQVRKAAITSYEKSDGVYTGLSTRNQETYETLKHEKPPQ

SEQ ID NO: 59 - DNA, 993 bp, Artificial Sequence
  CAR comprsing CD64c ECD - CD8 - CD28 TM - FceRIg domains
  misc_feature (1)..(993): CAR comprising CD64c ECD - CD8 - CD28 TM - FceRIg domains
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggccactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcaagggaaagcatcactacacatcagcaggaatatcacaatacactgtgaaaggcctccagttaccaactcctgtctggtttcatctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 60 - protein, 331 aa, Artificial Sequence
  CAR comprising CD64c ECD - CD8 - CD28 TM - FceRIg domains
  MISC_FEATURE (1)..(331): CAR comprising CD64c ECD - CD8 - CD28 TM - FceRIg domains
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWPLLQVSSRVFTEGEPLALRCHAWKDKLVYNVLYYRNGKAFKFFHWNSNLTILKTNISHNGTYHCSGKGKHHYTSAGISQYTVKGLQLPTPVWFHLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 61 - DNA, 840 bp, Artificial Sequence
  CAR comprising CD32a ECD - CD32a TM - FceRIg domains
  misc_feature (1)..(840): CAR comprising CD32a ECD - CD32a TM - FceRIg domains
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctacctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 62 - protein, 280 aa, Artificial Sequence
  CAR comprising CD32a ECD - CD32a TM - FceRIg domains
  MISC_FEATURE (1)..(280): CAR comprising CD32a ECD - CD32a TM - FceRIg domains
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYLKIQVRKAAITSYEKSDGVYTGLSTRNQETYETLKHEKPPQ

SEQ ID NO: 63 - DNA, 849 bp, Artificial Sequence
  CAR comprsing CD32a ECD - CD28 TM - FceRIg domains
  misc_feature (1)..(849): CAR comprising CD32a ECD - CD28 TM - FceRIg domains
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccattttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 64 - protein, 283 aa, Artificial Sequence
  CAR comprising CD32a ECD - CD28 TM - FceRIg domains
  MISC_FEATURE (1)..(283): CAR comprising CD32a ECD - CD28 TM - FceRIg domains
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPFWVLVVVGGVLACYSLLVTVAFIIFWVRLKIQVRKAAITSYEKSDGVYTGLSTRNQETYETLKHEKPPQ

SEQ ID NO: 65 - DNA, 1041 bp, Artificial Sequence
  CAR comprising CD32a ECD - CD8 - CD28 TM - FceRIg domains
  misc_feature (1)..(1041): CAR comprising CD32a ECD - CD8 - CD28 TM - FceRIg domains
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccactgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 66 - protein, 347 aa, Artificial Sequence
  CAR comprising CD32a ECD - CD8 - CD28 TM - FceRIg domains
  MISC_FEATURE (1)..(347): CAR comprising CD32a ECD - CD8 - CD28 TM - FceRIg
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 67 - DNA, 861 bp, Artificial Sequence
  CAR comprising CD32b ECD - CD32b TM - FceRIg domains
  misc_feature (1)..(861): CAR comprising CD32b ECD - CD32b TM - FceRIg domains
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctacctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 68 - protein, 287 aa, Artificial Sequence
  CAR comprsing CD32b ECD - CD32b TM - FceRIg domains
  MISC_FEATURE (1)..(287): CAR comprising CD32b ECD - CD32b TM - FceRIg domains
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYLKIQVRKAAITSYEKSDGVYTGLSTRNQETYETLKHEKPPQ

SEQ ID NO: 69 - DNA, 858 bp, Artificial Sequence
  CAR comprising CD32b ECD - CD28 TM - FceRIg domains
  misc_feature (1)..(858): CAR comprising CD32b ECD - CD28 TM - FceRIg
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 70 - protein, 286 aa, Artificial Sequence
  CAR comprising CD32b ECD - CD28 TM - FceRIg domains
  MISC_FEATURE (1)..(286): CAR comprising CD32b ECD - CD28 TM - FceRIg domains
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPFWVLVVVGGVLACYSLLVTVAFIIFWVRLKIQVRKAAITSYEKSDGVYTGLSTRNQETYETLKHEKPPQ

SEQ ID NO: 71 - DNA, 1050 bp, Artificial Sequence
  CAR comprising CD32b ECD - CD8 - CD28 TM - FceRIg domains
  misc_feature (1)..(1050): CAR comprising CD32b ECD - CD8 - CD28 TM - FceRIg domains
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 72 - protein, 350 aa, Artificial Sequence
  CAR comprising CD32b ECD - CD8 - CD28 TM - FceRIg domains
  MISC_FEATURE (1)..(350): CAR comprising CD32b ECD - CD8 - CD28 TM - FceRIg domains
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPLSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 73 - DNA, 69 bp, Artificial Sequence
  CD16A transmembrane domain
  misc_feature (1)..(69): CD16A transmembrane domain
taccaggtgtccttctgcctcgtgatggtgctgctgttcgccgtggacaccggcctgtacttcagcgtg

SEQ ID NO: 74 - protein, 23 aa, Artificial Sequence
  CD16A transmembrane domain
  MISC_FEATURE (1)..(23): CD16A transmembrane domain
YQVSFCLVMVLLFAVDTGLYFSV

SEQ ID NO: 75 - DNA, 69 bp, Artificial Sequence
  CD32A transmembrane domain
  misc_feature (1)..(69): CD32A transmembrane domain
atcattgtggctgtggtcattgcgactgctgtagcagccattgttgctgctgtagtggccttgatctac

SEQ ID NO: 76 - protein, 23 aa, Artificial Sequence
  CD32A transmembrane domain
  MISC_FEATURE (1)..(23): CD32A transmembrane domain
IIVAVVIATAVAAIVAAVVALIY

SEQ ID NO: 77 - DNA, 87 bp, Artificial Sequence
  CD32B transmembrane domain
  misc_feature (1)..(87): CD32B transmembrane domain
agctcttcaccgatggggatcattgtggctgtggtcactgggattgctgtagcggccattgttgctgctgtagtggccttgatctac

SEQ ID NO: 78 - protein, 29 aa, Artificial Sequence
  CD32B transmembrane domain
  MISC_FEATURE (1)..(29): CD32B transmembrane domain
SSSPMGIIVAVVTGIAVAAIVAAVVALIY

SEQ ID NO: 79 - DNA, 66 bp, Artificial Sequence
  CD64A transmembrane domain
  misc_feature (1)..(66): CD64A transmembrane domain
gtctggtttcatgtccttttctatctggcagtgggaataatgtttttagtgaacactgttctctgg

SEQ ID NO: 80 - protein, 22 aa, Artificial Sequence
  CD64A transmembrane domain
  MISC_FEATURE (1)..(22): CD64A transmembrane domain
VWFHVLFYLAVGIMFLVNTVLW

SEQ ID NO: 81 - DNA, 75 bp, Artificial Sequence
  CD64B transmembrane domain
  misc_feature (1)..(75): CD64B transmembrane domain
gtctggtttcatgtccttttctatctggcagtgggaataatgtttttagtgaacactgttctctgggtgacaata

SEQ ID NO: 82 - protein, 25 aa, Artificial Sequence
  CD64B transmembrane domain
  MISC_FEATURE (1)..(25): CD64B transmembrane domain
VWFHVLFYLAVGIMFLVNTVLWVTI

SEQ ID NO: 83 - DNA, 66 bp, Artificial Sequence; identical to SEQ ID NO: 79
  CD64C transmembrane domain
  misc_feature (1)..(66): CD64C transmembrane domain

SEQ ID NO: 84 - protein, 22 aa, Artificial Sequence
  CD64C transmembrane domain
  MISC_FEATURE (1)..(22): CD64C transmembrane domain
VWFHVLFYLAVGIMFLVNTVLW